(19) United States
(12) Patent Application Publication
(10) Pub. No.: US 2008/0019929 A1
(43) Pub. Date: Jan. 24, 2008

(54) Micro-Particulate Organic Uv Absorber Composition

(76) Inventors: Cyrille Deshayes, Rosenau (FR); Sebastien Mongiat, Sierentz (FR)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 11/661,173

(22) PCT Filed: Aug. 29, 2005
(86) PCT No.: PCT/EP05/54225

§ 371 Date: Feb. 26, 2007

(30) Foreign Application Priority Data
Sep. 1, 2004 (EP) 04104185.6

(51) Int. Cl. A61K 8/00; A61Q 17/00

(52) U.S. Cl. 424/59

(57) ABSTRACT

Disclosed is a topically applicable water-in-oil (w/o) or water-in-silicone (w/Si) formulation comprising: (a) at least one aqueous phase; (b) at least one fatty phase; (c) an effective UV-photoprotecting composition comprising (c₁) 20% to 99% of at least one insoluble micronized organic UV-screening active with a mean particle size ranging from 0.01 μm to 2 μm; and (c₂) 0.5% to 80% of at least one primary C₆-C₂₈ fatty alcohol. The combination of a micronized organic UV absorber and a specific fatty alcohol allows a maximum of miscibility of micronized organic particles within the continuous oil phase of w/o emulsion wherein the micronized particles are stabilized in the oil phase of the composition. Simultaneously the crystal growth of micronized organic UV absorbers within W/O emulsions is limited or even eliminated.

The present invention relates to topically applicable water-in-oil (w/o) or water-in-silicone (w/Si) formulations, in particular, to new UV-protection formulations which comprise a micronised UV absorber, wherein the micronised UV absorber is combined with specific fatty acid alcohol.

It has long been known that prolonged exposure to UV radiation can lead to the formation of erythemas or light dermatoses, as well as to an increased incidence of skin cancers, or accelerated skin ageing.

Various sunscreen formulations have been proposed which include materials which are intended to counteract UV radiation, thereby inhibiting the said undesired effects on the skin.

A great number of compounds have been proposed for use as UV protectants in sunscreen formulations, especially soluble organic UV absorbers and insoluble micronised inorganic compounds, in particular zinc oxide and titanium dioxide.

With respect to the use in sunscreen formulations of soluble organic UV absorbers, they have the disadvantages that their effectiveness as UV protectants in terms of SPF (Sun Protection Factor) in a sunscreen formulation is often too low for commercial purposes; as a result of their solubility, they exhibit relatively high allergenic potential; and that as a result of intrinsic photochemical lability, the duration of the protective effect is often too low.

The high specific weight of insoluble inorganic compounds, such as titanium dioxide leads to a reduced stability of formulations containing them. Moreover, such inorganic compounds have been claimed to generate toxic radicals under the influence of light and water (“Redox Mechanisms in Heterogeneous Photocatalysis”, Serpone et al, Electrochemistry in Colloids and Dispersions, Editors Mackay and Texter, VCH Publishers Inc., New York 1992).

In GB-A-2303549, there is described a method of producing micronised, insoluble organic UV absorbers, as well as a sunscreen composition comprising a micronised formulation of an insoluble organic UV absorber, produced according to the said method.

Micronised insoluble organic When the so obtained UV absorbers are used in sunscreen formulations they provide excellent UV protection and have an SPF rating which is at least as high as corresponding sunscreen formulations containing a known inorganic UV absorber. Unlike the latter UV absorbers micronised insoluble organic UV absorbers show no tendency for generating radicals which could damage or sensitise human skin under the influence of light.

In WO 99/66896 a micronised organic UV absorber sun screen composition is disclosed, wherein the micronised organic UV absorber is present in the oil phase of the formulation.

Surprisingly it was found that the combination of a micronized organic UV absorber and a specific fatty alcohol allows a maximum of miscibility of micronized organic particles within the continuous oil phase of w/o emulsions wherein the micronized particles are stabilized in the oil phase of the composition. Simultaneously the crystal growth of micronized organic UV absorbers within W/O emulsions is limited or even eliminated.

Therefore, the present invention relates to a topically applicable water-in-oil (w/o) or water-in-silicone (w/Si) formulation comprising:
(a) at least one aqueous phase;
(b) at least one fatty phase;
(c) an effective UV-photoprotecting composition comprising
   (c₁) 20% to 99% of at least one insoluble micronized organic UV-screening active with a mean particle size ranging from 0.01 μm to 2 μm; and
   (c₂) 0.5% to 80% of at least one primary C₆-C₂₈ fatty alcohol.

Suitable organic UV absorber (c₁) may be, e.g. a triazine, a benzotriazole, a benzophenone, a vinyl group-containing amide, a cinnamic acid amide or a sulfonated benzimidazole UV absorber.

A preferred class of triazine compounds is that having the formula 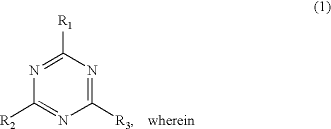
R₁, R₂ and R₃, independently from each other, are hydrogen; hydroxy; C₁-C₃alkoxy; NH₂; NHR₄; N(R₄)₂; OR₄; C₆-C₁₂aryl; phenoxy; anilino; pyrrolo; in which the respective phenyl, phenoxy, anilino or pyrrolo moieties are not substituted or substituted by one, two or three substitutents selected from OH, carboxy, CO—NH₂, C₁-C₁₂alkyl, C₁-C₁₂alkoxy, a methylidenecamphor group, a group —(CH═CH)ₘC(═O)—OR₄, a group 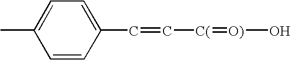
   or the corresponding alkali metal, ammonium, mono-, di- or tri-C₁-C₄alkylammonium, mono-, di- or tri-C₂-C₄alkanolammonium salts, or the C₁-C₃alkyl esters thereof or by a radical of formula 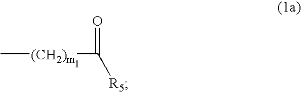
R₄ is C₁-C₅alkyl;
R₅ is hydroxy; C₁-C₅alkyl that is unsubstituted or substituted by one or more OH groups; C₁-C₅alkoxy; amino; mono- or di-C₁-C₅alkylamino; M; a radical of formula 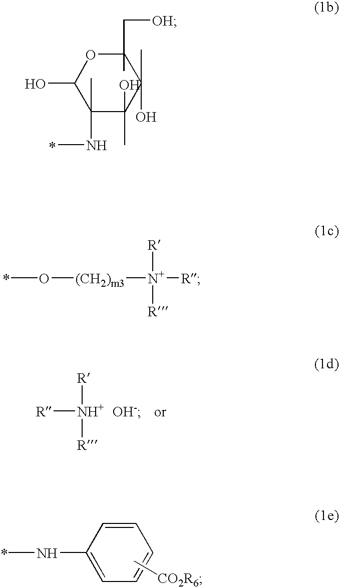
R′, R″ and R′″ independently of the other are C₁-C₁₄alkyl that is unsubstituted or substituted by one or more OH groups;
R₆ is hydrogen; M; C₁-C₅alkyl; or a radical of the formula —(OH₂)ₘ_{₂}—O-T₁;
M is a metal cation;
T₁ is hydrogen; or C₁-C₈alkyl;
m is 0 or 1;
m₁ is from 1 to 5;
m₂ is from 1 to 4; and
m₃ is from 2 to 14.

Preferred compounds of formula (1) are those, wherein
R₁, R₂ and R₃ independently from each other are a radical of formula 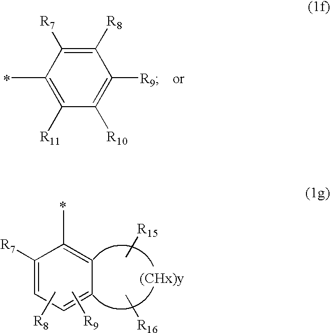
R₇ and R₁₁ independently from each other are hydrogen; C₁-C₁₈alkyl; or C₆-C₁₂aryl;
R₈, R₉ and R₁₀, independently from each other, are hydrogen; or a radical of formula 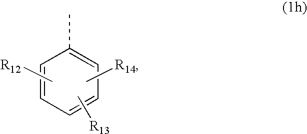
   wherein, in formula (1f), at least one of the radicals R₈, R₉ and R₁₀ are a radical of formula (1h);
R₁₂, R₁₃, R₁₄, R₁₅ and R₁₆ independently from each other are hydrogen; hydroxy; halogen; C₁-C₁₈alkyl; C₁-C₁₈alkoxy; C₆-C₁₂aryl; biphenylyl; C₆-C₁₂aryloxy; C₁-C₁₈alkylthio; carboxy; —COOM; C₁-C₁₈-alkylcarboxyl; aminocarbonyl; or mono- or di-C₁-C₁₈alkylamino; C₁-C₁₀acylamino; —COOH;
M is an alkali metal ion;
x is 1 or 2; and
y is a number from 2 to 10.

Most preferred triazine derivatives are compounds of formula 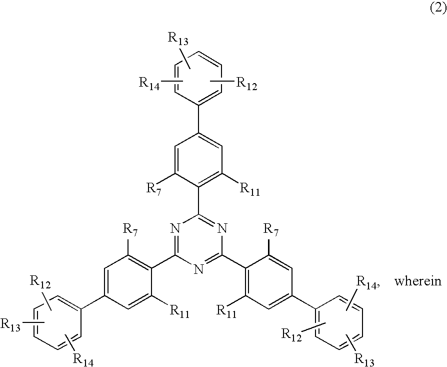
R₇, R₁₁, R₁₂, R₁₃ und R₁₄ are defined as in formula (1f), (1g) or (1h), and most preferably compound of formula (2), wherein
R_{7 and} R₁₁ are hydrogen.

Furthermore, triazine derivatives of formula 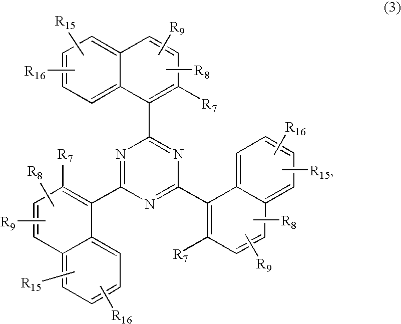
are preferred, wherein
R₇, R₈, R₉, R₁₅ and R₁₆ are defined as in formula (1g), and most preferably compounds of formula (3), wherein
R₇, R₈, R₉, R₁₅ and R₁₆ are hydrogen; or, independently from each other, C₁-C₁₈alkyl.

Most preferred as component (c₁) are triazine derivatives of formula

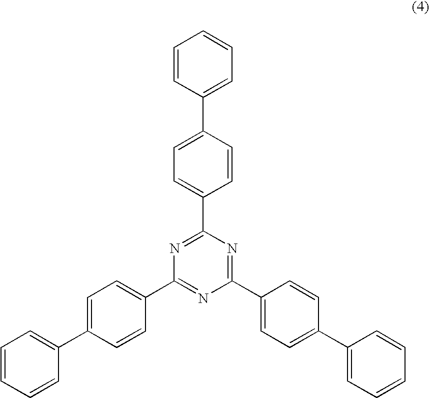

Further preferred triazine derivatives according to component (c₁) correspond to formula 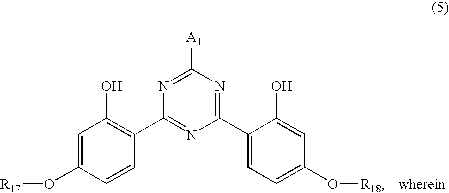
R₁₇ and R₁₈, independently of one another, are C₁-C₁₈alkyl; C₂-C₁₈alkenyl; a radical of the formula —CH₂—CH(—OH)—CH₂—O-T₁; or a radical of the formula —(CH₂)ₘ_{₁}—O—(CH₂)ₘ_{₂}-T₂; a radical of the formula 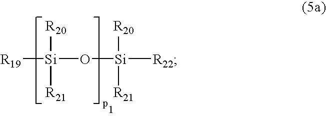
R₁₉ is a direct bond; a straight-chain or branched C₁-C₄alkylene radical or a radical of the formula —Cₘ_{₁}H₂ₘ_{₁}— or —Cₘ_{₁}H₂ₘ_{₁}—O—;
R₂₀, R₂₁ and R₂₂, independently of one another, are C₁-C₁₈alkyl; C₁-C₁₈alkoxy or a radical of the formula 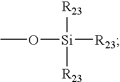
R₂₃ is C₁-C₅alkyl;
T₁ and T₂, independently from each other, are hydrogen; or C₁-C₈alkyl;
m₁, m₂ and m₃, independently of one another, are 1 to 4;
p₁ is 0; or a number from 1 to 5;
A₁ is a radical of the formula 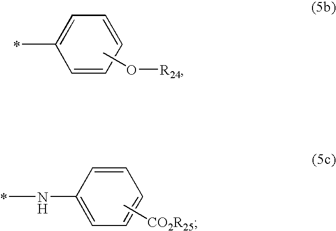
   or of the formula 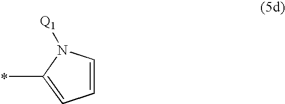
R₂₄ is hydrogen; C₁-C₁₀alkyl, —(CH₂CHR₂₆—O)ₙ_{₁}—R₂₅; a —CH₂—CH(—OH)—CH₂—O-T₁; or radical of the formula —(CH₂)ₘ_{₁}—O—(CH₂)ₘ_{₂}-T₂
R₂₅ is hydrogen; M; C₁-C₅alkyl; or a radical of the formula —(CH₂)ₘ_{₂}—O-T₁;
R₂₆ is hydrogen; or methyl;
Q₁ C₁-C₁₈alkyl;
M is a metal cation; and
n₁ is 1-16.

Further preferred triazine derivatives according to component (c₁) are compounds of formulae 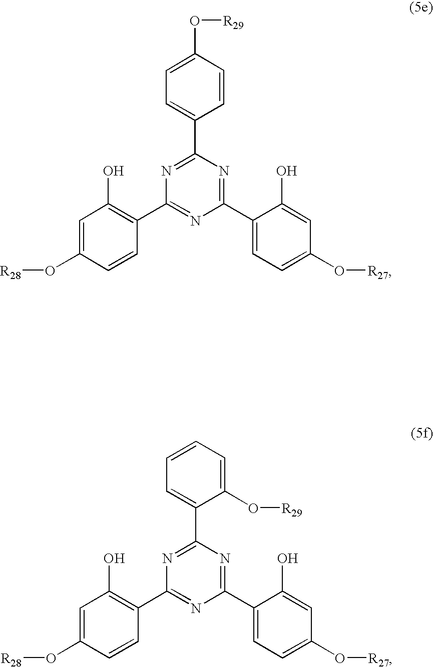
the formula 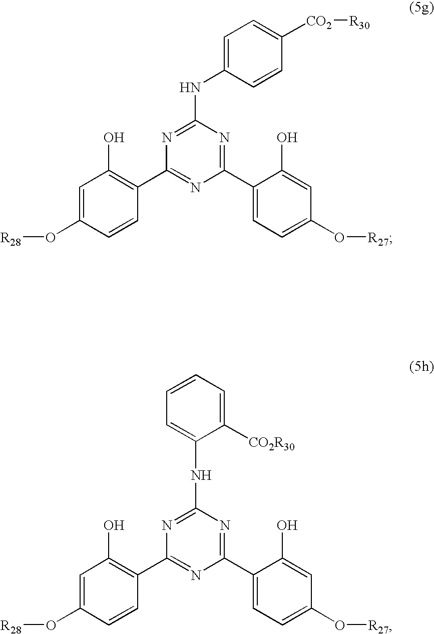
in which
R₂₇ and R₂₈, independently of one another, are C₃-C₁₈alkyl; or —CH₂—CH(—OH)—CH₂—O-T₁;
R₃₀ is C₁-C₁₀alkyl or a radical of the formula 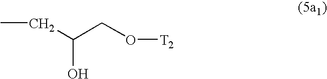
   or the formula 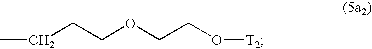
R₃₀ is hydrogen; M; C₁-C₅alkyl; —NH—C₁-C₅alkyl, preferably —NH-tert.alkyl; or a radical of the formula —(CH₂)ₘ—O-T₂;
T₁ and T₂, independently of one another, are hydrogen; or C₁-C₅alkyl; and
m is 1 to 4.

Uppermost of interest are compounds of the formulae (5e) and (5f), in which
R₂₇ and R₂₈, independently of one another, are C₃-C₁₈alkyl; or —CH₂—CH(—OH)—CH₂—O-T₁;
R₂₉ is C₁-C₁₀alkyl;
and compounds of the formulae (5g) and (5h), in which
R₂₇ and R₂₈, independently of one another, are C₃-C₁₈alkyl or —CH₂—CH(—OH)—CH₂—O-T₁; and
T₁ is hydrogen; or C₁-C₅alkyl.

Very particularly preferred in this case are triazine compounds of the formula (5e)-(5h), in which R₂₇ and R₂₈ have the same meaning.

Furthermore, interesting triazines correspond to the formula 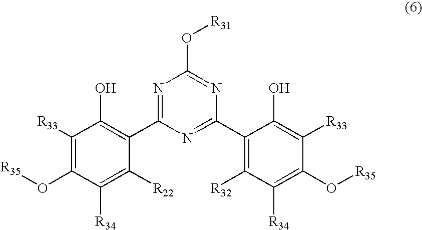
in which
R₃₁ is C₁-C₃₀alkyl; C₂-C₃₀alkenyl; unsubstituted or C₁-C₅alkyl-mono- or polysubstituted C_{5-C}₁₂cycloalkyl, C₁-C₅alkoxy-C₁-C₁₂alkyl; amino-C₁-C₁₂alkyl; C₁-C₅monoalkylamino-C_{1-C}₁₂alkyl; C₁-C₅dialkylamino-C₁-C₁₂alkyl; a radical of the formula 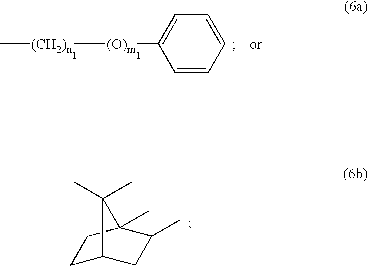
R₃₂, R₃₃ and R₃₄, independently of one another, are hydrogen; hydroxyl; C₁-C₃₀alkyl; or C₂-C₃₀alkenyl;
R₃₅ is hydrogen; or C₁-C₅alkyl;
m₁ is 0 or 1; and
n₁ is 1 to 5.

Preferred compounds correspond to the formula 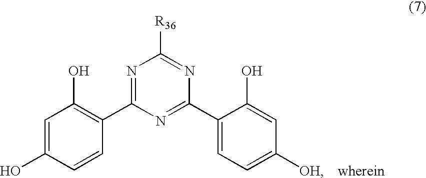
R₃₆ is

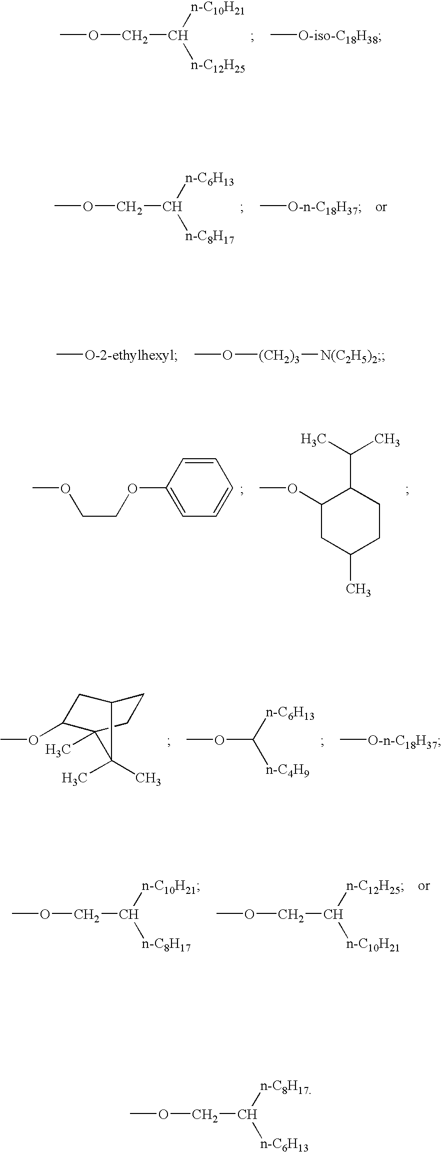

Further preferred triazine derivatives according to component (c₁) are those compounds having one of the formulae 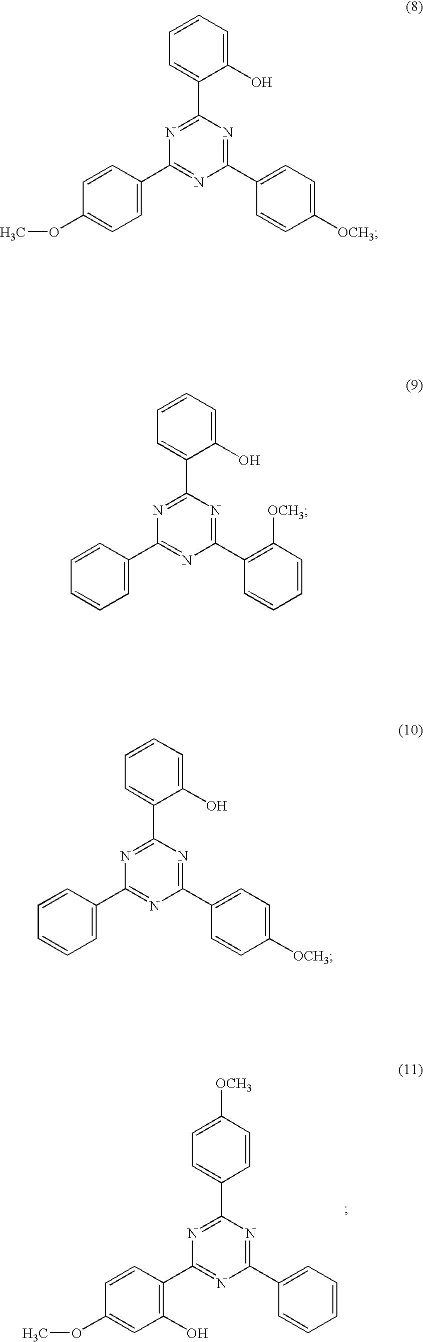 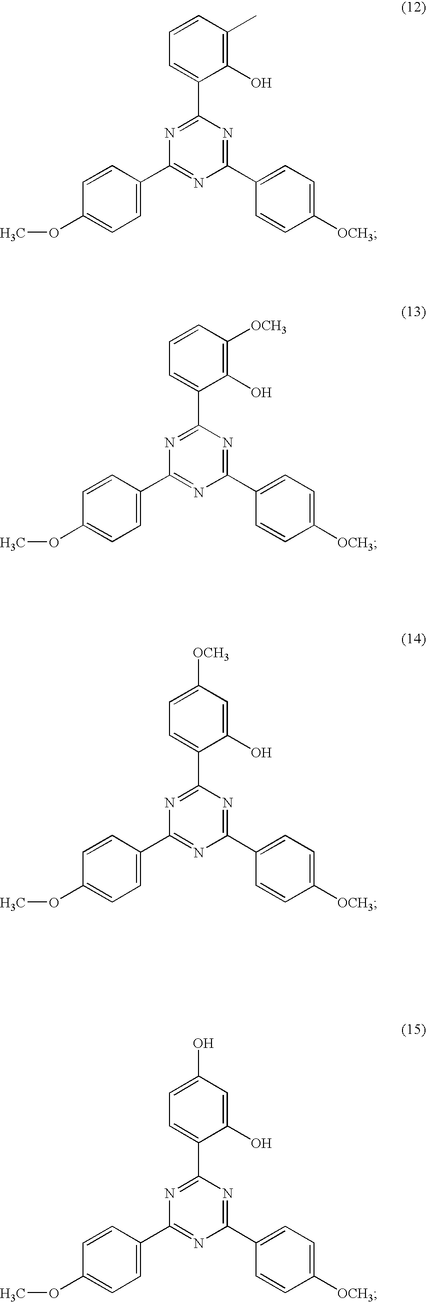 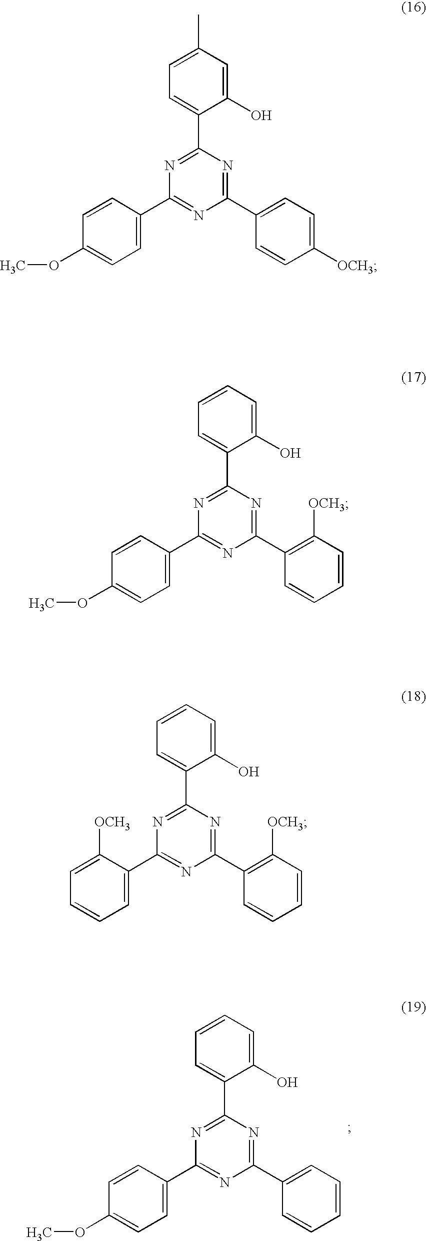 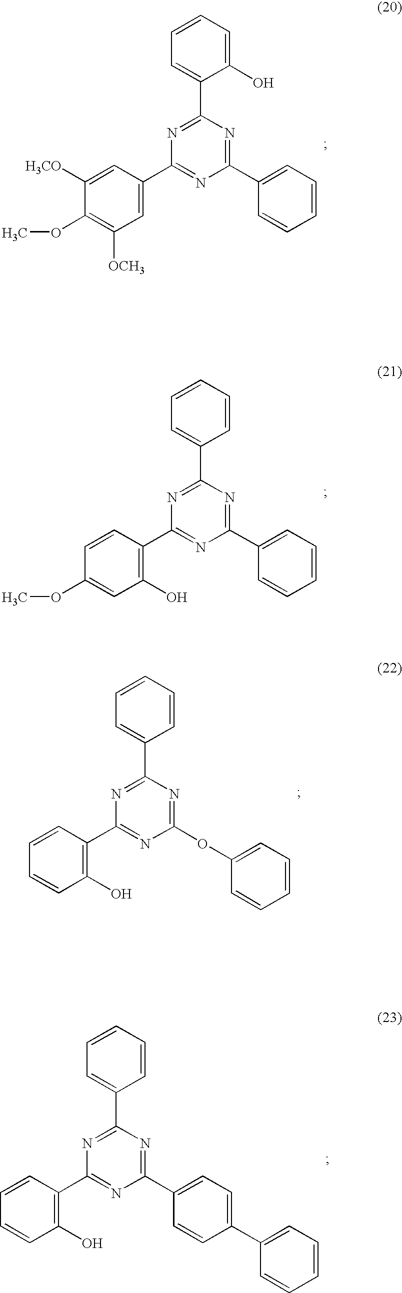 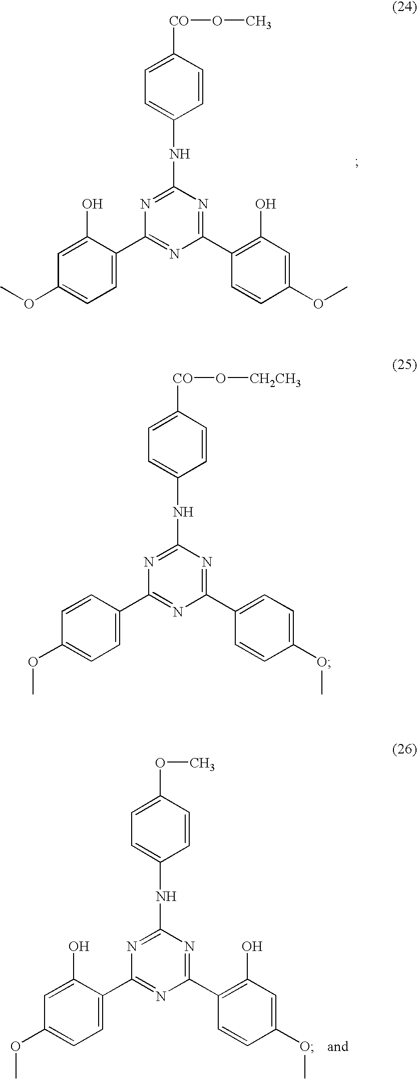 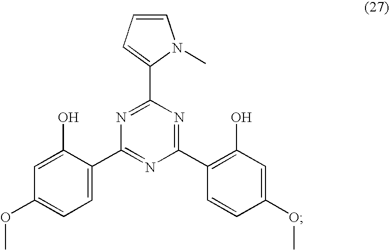
as well as 2,4,6-tris(diisobutyl-4′-aminobenzalmalonate)-s-triazine and 2,4-bis(diisobutyl-4-aminobenzalmalonate)-6-(4′-aminobenzylidenecamphor)-s-triazine.

Particularly preferred compounds of formula (1) are those having the formula: 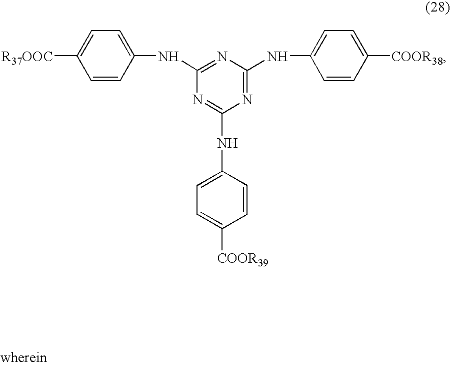
R₃₇, R₃₈ and R₃₉, independently from each other are hydrogen; an alkali metal; or an ammonium group N⁺(R₄₀)₄;
R₄₀ is hydrogen; or an organic radical; C₁-C₃alkyl; or a polyoxyethylene radical which contains from 1 to 10 ethylene oxide units and the terminal OH group of which may be etherified by a C₁-C₃alcohol.

In relation to the compounds of formula (28), when R₃₇, R₃₈ and R₃₉ is an alkali metal it is preferably potassium or, especially sodium; when R₃₇, R₃₈ and R₃₉ is a group N(R₄₀)₄ in which R₃₀ has its previous significance, it is preferably a mono-, di- or tri-C₁-C₄alkylammonium salt, a mono-, di- or tri-C₂-C₄alkanolammonium salt or a C₁-C₃alkyl ester thereof; when R₄₀ is a C₁-C₃alkyl group, it is preferably a C₁-C₂alkyl group, more preferably a methyl group; and when R₃₀ is polyoxyethylene group, this preferably contains from 2-6 ethylene oxide units.

One preferred class of triazole micronised organic UV absorbers is that having the formula 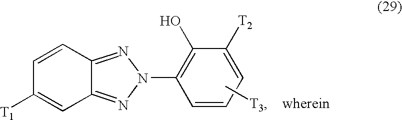
T₁ is C₁-C₃alkyl or, preferably, hydrogen; or a radical of formula 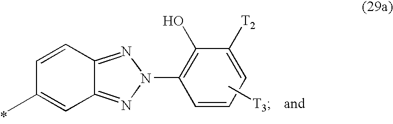
T₂ and T₃, independently from each other are C₁-C₁₂alkyl, preferably i-octyl; or C₁-C₄alkyl substituted by phenyl, preferably α,α-dimethylbenzyl.

A further preferred class of triazole micronised organic UV absorbers corresponds to the formula 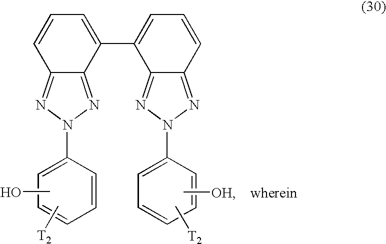
T₂ has its previous significance.

A still further preferred class of triazole micronised organic UV absorbers corresponds to the formula 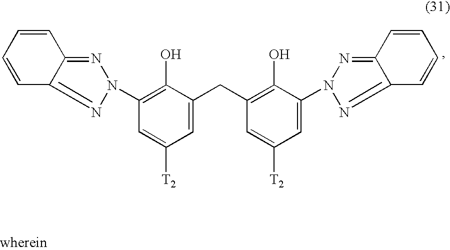
T₂ is hydrogen; C₁-C₁₂alkyl, preferably iso-octyl, or C₁-C₄alkyl substituted by phenyl, preferably α,α-dimethylbenzyl.

A preferred class of vinyl group-containing amide micronised organic UV absorbers corresponds to the formula:
(32) R₄₁—(Y)ₘ—CO—C(R₄₂)═C(R₄₃)—N(R₄₄)(R₄₅), wherein
R₄₁ is C₁-C₃alkyl, preferably C₁-C₂alkyl, or phenyl optionally substituted by one, two or three substituents selected from OH, C₁-C₃alkyl, C₁-C₃alkoxy or CO—OR₄₆,
R₄₆ C₁-C₃alkyl;
R₄₂, R₄₃, R₄₄ and R₄₅ are the same or different and each is C₁-C₃alkyl, preferably C₁-C₂alkyl; or hydrogen;
Y is —NH—; or —O—; and
m is 0; or 1.

Preferred compounds of formula (32) are 4-methyl-3-penten-2-one, ethyl-3-methylamino-2-butenoate, 3-methylamino-1-phenyl-2-buten-1-one and 3-methylamino-1-phenyl-2-buten-1-one.

A preferred class of cinnamic acid amide micronised organic UV absorbers corresponds to the formula: 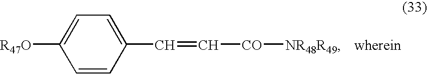
R₄₇ is hydroxy or C₁-C₄alkoxy, preferably methoxy or ethoxy;
R₄₈ is hydrogen or C₁-C₄alkyl, preferably methyl or ethyl; and
R₄₉ is —(CONH)ₘ-phenyl in which m is 0 or 1 and the phenyl group is optionally substituted by one, two or three substituents selected from OH, C₁-C₃alkyl, C₁-C₃alkoxy or CO—OR₅₀; and
R₅₀ is C₁-C₄alkyl.

A preferred class of sulfonated benzimidazole micronised organic UV absorbers corresponds to the formula 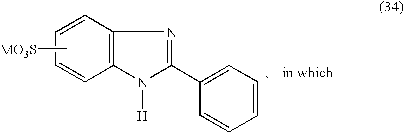
M is hydrogen; or an alkali metal, preferably sodium, an alkaline earth metal, such as magnesium or calcium, or zinc.

Further preferred classes of micronised or micronisable UV absorbers used for the present invention are:
p-aminobenzoic acid derivatives, typically 2-ethylhexyl-4-dimethylaminobenzoate
salicylic acid derivatives, typically 2-ethylhexyl salicylate; homosalates; and isopropyl sylicylates;
benzophenone derivatives, typically 2-hydroxy-4-methoxybenzophenone;
dibenzoylmethane derivatives, typically 1-(4-tert-butylphenyl)-3-(4-methoxyphenyl)-propane-1,3-dione;
diphenylacrylates, typically 2-ethylhexyl-2-cyano-3,3-diphenylacrylate and 3-(benzo-furanyl)-2-cyanoacrylate;
3-imidazol-4-yl-acrylic acid and 3-imidazol-4-yl-acrylate;
benzofurane derivatives, preferably 2-(p-aminophenyl)benzofuran derivatives, disclosed in EP-A-582 189, U.S. Pat. No. 5,338,539, U.S. Pat. No. 5,518,713 and EP-A-613 893;
polymeric UV absorbers, such as the benzylidenemalonate derivatives described, inter alia in EPA-709 080;
cinnamic acid derivatives, typically the 2-ethylhexyl-4-methoxycinnamate or isoamylate or cinnamic acid derivatives disclosed, inter alia, in U.S. Pat. No. 5,601,811 and WO 97/00851;
camphor derivatives, typically 3-(4′-methyl)benzylidenebornan-2-one, 3-benzylidene-bornan-2-one, N-[2(and 4)-2-oxyborn-3-ylidenemethyl)benzyl]acrylamide polymer, 3-(4′-trimethylammonium)benzylidenebornan-2-one methylsulfate, 3,3′-(1,4-phenylenedime-thine)-bis(7,7-dimethyl-2-oxobicyclo-[2.2.1]heptane-1-methanesulfonic acid) and the salts thereof, 3-(4′-sulfo)benzylidenebornan-2-one and the salts thereof;
2-phenylbenzimidazole-5-sulfonic acids and the salts thereof; and
menthyl-o-aminobenzoate.

Fatty alcohols are generally understood as saturated or unsaturated primary alcohols (1-al-kanols) having 6 to 22 carbon atoms. They are obtained by reduction of triglycerides, fatty acids or fatty acid methyl esters.

Preferably the fatty alcohol according to component (c₂) is a linear or linear branched C₈-C₂₀alcohol.

The fatty alcohols according to component (c₂) are preferably characterized by primary alcohols which correspond to the formula 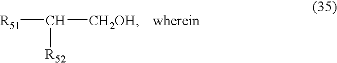
R₅₁ and R₅₂, independently from each other, are a linear C₁-C₁₈alkyl, wherein the sum of carbon atoms in R₅₁ and R₅₂ is ≦18.

Examples of fatty alcohols according to component (c₂) correspond to the formula

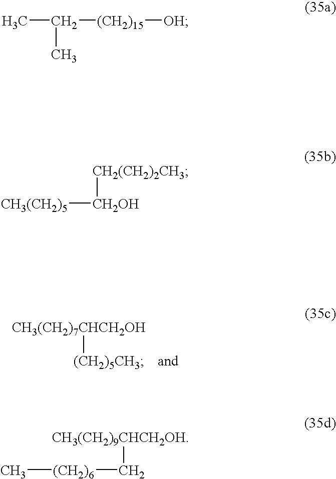

The fatty alcohols according to component (c₂) in the composition of the present invention may be used as single compounds or as mixtures of 2 or more than 2 single compounds.

Preferably, mixtures of linear octanol and linear decanol are used as component (c₂).

Preferred combinations of insoluble micronized organic UV-screening actives (component (c₁) and primary C₆-C₂₈ fatty alcohols (component (c₂)) are:

| insoluble micronized organic | |
|---|---|
| UV-screening active (C₁) | primary C₆-C₂₈ fatty alcohol (=c₂) |
| Compound of formula (31), wherein T₂ is isooctyl | 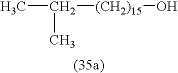 |
| Compound of formula (31), wherein T₂ is isooctyl | 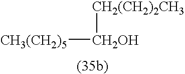 |
| Compound of formula (31), wherein T₂ is isooctyl | 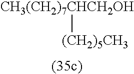 |
| Compound of formula (31), wherein T₂ is isooctyl | 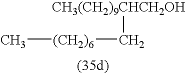 |
| Compound of formula (31), wherein | mixture of linear octanol and linear decanol |
| T₂ is isooctyl | |
| Compound of formula (4) | 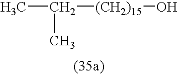 |
| Compound of formula (4) | 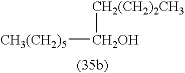 |
| Compound of formula (4) | 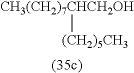 |
| Compound of formula (4) | 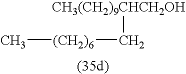 |
| Compound of formula (4) | mixture of linear octanol and linear decanol |

The micronised organic UV absorber, component (c₁), is preferably produced by the method described in GB-A-2303549, namely by a process which comprises grinding the corresponding organic UV absorber, in coarse particle form, in a grinding apparatus, in the presence of 1 to 50%, preferably 5 to 40% by weight, based on the micronised organic UV absorber, of an alkyl polyglucoside having the formula CₙH₂ₙ₊₁O(C₆H₁₀O₅)ₓH, in which n is an integer ranging from 8 to 16 and x is the mean polymerisation level of the glucoside moiety (C₆H₁₀O₅) and ranges from 1.4 to 1.6, or an ester thereof.

Any known process suitable for the preparation of microparticles can be used for the preparation of the micronised UV absorbers, for example wet-milling, wet-kneading, spray-drying from a suitable solvent, by the expansion according to the RESS process (Rapid Expansion of Supercritical Solutions), by reprecipitation from suitable solvents, including supercritical fluids (GASR process=Gas Anti-Solvent Recrystallisation/PCA process=Precipitation with Compressed Anti-solvents).

The micronised UV absorbers so obtained usually have an average particle size from 0.02 to 2, preferably from 0.03 to 1.5, and more especially from 0.05 to 1.0 micrometer.

The UV absorbers according to component (c₁) can also be used as dry substrates in powder form

The topically applicable water-in-oil (w/o) or water-in-silicone (w/Si) formulation according to the present invention may additionally contain one or more than one further non-micronized UV filter as listed in Tables 1 and 2.

The UV absorbers as described in Tables 1 and 2 below may be added to the topically applicable water-in-oil (w/o) or water-in-silicone (w/Si) formulations according to the present invention in a amounts from 0.1 to 20% b.w. Mixtures of these UV absorbers can be used, inter alia, to improve the solubility or to increase UV absorption.

TABLE 1
Suitable non-micronized UV filter substances which can be additionally
used with the UV absorbers according to the present invention

p-aminobenzoic acid derivatives, for example 4-dimethylaminobenzoic acid 2-ethylhexyl
ester;
salicylic acid derivatives, for example salicylic acid 2-ethylhexyl ester;
benzophenone derivatives, for example 2-hydroxy-4-methoxybenzophenone and its
5-sulfonic acid derivative;
dibenzoylmethane derivatives, for example 1-(4-tert-butylphenyl)-3-(4-methoxyphenyl)-
propane-1,3-dione;
diphenylacrylates, for example 2-ethylhexyl 2-cyano-3,3-diphenylacrylate, and 3-(benzo-
furanyl)2-cyanoacrylate;
3-imidazol-4-ylacrylic acid and esters;
benzofuran derivatives, especially 2-(p-aminophenyl)benzofuran derivatives, described in
EP-A-582 189, US-A-5 338 539, US-A-5 518 713 and EP-A-613 893;
polymeric UV absorbers, for example the benzylidene malonate derivatives described in
EP-A-709 080;
cinnamic acid derivatives, for example the 4-methoxycinnamic acid 2-ethylhexyl ester and
isoamyl ester or cinnamic acid derivatives described in US-A-5 601 811 and WO 97/00851;
camphor derivatives, for example 3-(4′-methyl)benzylidene-bornan-2-one, 3-benzylidene-
bornan-2-one, N-[2(and 4)-2-oxyborn-3-ylidene-methyl)-benzyl]acrylamide polymer, 3-(4′-
trimethylammonium)-benzylidene-bornan-2-one methyl sulfate, 3,3′-(1,4-
phenylenedimethine)-bis(7,7-dimethyl-2-oxo-bicyclo[2.2.1]heptane-1-methanesulfonic acid)
and salts, 3-(4′-sulfo)benzylidene-bornan-2-one and salts; camphorbenzalkonium
methosulfate;
hydroxyphenyltriazine compounds, for example 2-(4′-methoxyphenyl)-4,6-bis(2′-hydroxy-4′-
n-octyloxyphenyl)-1,3,5-triazine; 2,4-bis{[4-(3-(2-propyloxy)-2-hydroxy-propyloxy)-2-
hydroxy]-phenyl}-6-(4-methoxyphenyl)-1,3,5-triazine; 2,4-bis{[4-(2-ethyl-hexyloxy)-2-
hydroxy]-phenyl}-6-[4-(2-methoxyethyl-carboxyl)-phenylamino]-1,3,5-triazine; 2,4-bis{[4-
(tris(trimethylsilyloxy-silylpropyloxy)-2-hydroxy]-phenyl}-6-(4-methoxyphenyl)-1,3,5-triazine;
2,4-bis{[4-(2″-methylpropenyloxy)-2-hydroxy]-phenyl}-6-(4-methoxyphenyl)-1,3,5-triazine;
2,4-bis{[4-(1′,1′,1′,3′,5′,5′,5′-heptamethyltrisilyl-2″-methyl-propyloxy)-2-hydroxy]-phenyl}-6-
(4-methoxyphenyl)-1,3,5-triazine; 2,4-bis{[4-(3-(2-propyloxy)-2-hydroxy-propyloxy)-2-
hydroxy]-phenyl}-6-[4-ethylcarboxy)-phenylamino]-1,3,5-triazine;
benzotriazole compounds, for example 2,2′-methylene-bis(6-(2H-benzotriazol-2-yl)-4-
(1,1,3,3-tetramethylbutyl)-phenol;
trianilino-s-triazine derivatives, for example 2,4,6-trianiline-(p-carbo-2′-ethyl-1′-oxy)-1,3,5-
triazine and the UV absorbers disclosed in US-A-5 332 568, EP-A-517 104, EP-A-507 691,
WO 93/17002 and EP-A-570 838;
2-phenylbenzimidazole-5-sulfonic acid and salts thereof;
menthyl o-aminobenzoates;
physical sunscreens coated or not as titanium dioxide, zinc oxide, iron oxides, mica, MnO,
Fe₂O₃, Ce₂O₃, Al₂O₃, ZrO₂. (surface coatings: polymethylmethacrylate, methicone
(methylhydrogenpolysiloxane as described in CAS 9004-73-3), dimethicone, isopropyl
titanium triisostearate (as described in CAS 61417-49-0), metal soaps as magnesium
stearate (as described in CAS 4086-70-8), perfluoroalcohol phosphate as C9-15
fluoroalcohol phosphate (as described in CAS 74499-44-8; JP 5-86984, JP 4-330007)).
The primary particle size is an average of 15 nm-35 nm and the particle size in dispersion is
in the range of 100 nm-300 nm.
aminohydroxy-benzophenone derivatives disclosed in DE 10011317, EP 1133980 and EP
1046391
phenyl-benzimidazole derivatives as disclosed in EP 1167358
the UV absorbers described in “Sunscreens”, Eds. N. J. Lowe, N. A. Shaath, Marcel Dekker,
Inc., New York and Basle or in Cosmetics & Toiletries (107), 50ff (1992) also can be used
as additional UV protective substances.

TABLE 2
Suitable specific non-micronized UV filter substances and adjuvants
which can be additionally used with the UV
absorbers according to the present invention

| No. | Chemical Name | CAS No. |
|---|---|---|
| 1 | (+/−)-1,7,7-trimethyl-3-[(4- | 36861-47-9 |
| | methylphenyl)methylene]bicyclo- | |
| | [2.2.1]heptan-2-one; p-methyl benzylidene camphor | |
| 2 | 1,7,7-trimethyl-3- | 15087-24-8 |
| | (phenylmethylene)bicyclo[2.2.1]heptan-2-one; | |
| | benzylidene camphor | |
| 3 | (2-Hydroxy-4-methoxyphenyl)(4- | |
| | methylphenyl)methanone | 1641-17-4 |
| 4 | 2,4-dihydroxybenzophenone | 131-56-6 |
| 5 | 2,2′,4,4′-tetrahydroxybenzophenone | 131-55-5 |
| 6 | 2-Hydroxy-4-methoxy benzophenone; | 131-57-7 |
| 7 | 2-Hydroxy-4-methoxy benzophenone-5- | 4065-45-6 |
| | sulfonic acid | |
| 8 | 2,2′-dihydroxy-4,4′-dimethoxybenzophenone | 131-54-4 |
| 9 | 2,2′-Dihydroxy-4-methoxybenzopheNone | 131-53-3 |
| 10 | Alpha-(2-oxoborn-3-ylidene)toluene-4-sulphonic | 56039-58-8 |
| | acid and its salts; Mexoryl SL | |
| 11 | 1-[4-(1,1-dimethylethyl)phenyl]-3-(4- | 70356-09-1 |
| | methoxyphenyl)propane-1,3-dione; avobenzone | |
| 12 | Methyl N,N,N-trimethyl-4-[(4,7,7-trimethyl-3- | 52793-97-2 |
| | oxobicyclo[2,2,1]hept-2- | |
| | ylidene)methyl]anilinium sulphate; Mexoryl SO | |
| 22 | 3,3,5-Trimethyl cyclohexyl-2-hydroxy benzoate; | 118-56-9 |
| | homosalate | |
| 23 | Isopentyl p-methoxycinnamate; isoamyl methoxy | 71617-10-2 |
| | cinnamate | |
| 27 | Menthyl-o-aminobenzoate | 134-09-8 |
| 28 | Menthyl salicylate | 89-46-3 |
| 29 | 2-Ethylhexyl 2-cyano,3,3-diphenylacrylate; | 6197-30-4 |
| | octocrylene | |
| 30 | 2- ethylhexyl 4- (dimethylamino)benzoate | 21245-02-3 |
| 31 | 2- ethylhexyl 4- methoxycinnamate; | 5466-77-3 |
| | octyl methoxy cinnamate | |
| 32 | 2- ethylhexyl salicylate | 118-60-5 |
| 33 | Benzoic acid, 4, 4′, 4″- (1, 3, 5-triazine- 2, 4, 6- | 88122-99-0 |
| | triyltriimino)tris-, tris(2-ethylhexyl)ester; 2,4,6- | |
| | Trianilino-(p-carbo-2′-ethylhexyl-1′-oxi)-1,3,5- | |
| | triazine; octyl triazone | |
| 34 | 4-aminobenzoic acid | 150-13-0 |
| 35 | Benzoic acid, 4-amino-, ethyl ester, polymer with | 113010-52-9 |
| | oxirane | |
| 38 | 2- phenyl- 1H- benzimidazole- 5-sulphonic acid; | 27503-81-7 |
| | phenylbenzimidazolsulfonic acid | |
| 39 | 2-Propenamide, N-[[4-[(4,7,7-trimethyl-3- | 147897-12-9 |
| | oxobicyclo[2.2.1]hept-2- | |
| | ylidene)methyl]phenyl]methyl]-, | |
| | homopolymer | |
| 40 | Triethanolamine salicylate | 2174-16-5 |
| 41 | 3, 3′-(1,4-phenylenedimethylene)bis[7, 7- | 90457-82-2 |
| | dimethyl- 2-oxo-bicyclo[2.2.1]heptane- | |
| | 1 methanesulfonic acid]; Cibafast H | |
| 42 | Titanium dioxide | 13463-67-7 |
| 44 | Zinc oxide | 1314-13-2 |
| 45 | 2,4-bis{[4-(2-ethylhexyloxy)-2-hydroxy]- | 187393-00-6 |
| | phenyl}-6-(4-methoxyphenyl)-(1,3,5)-triazine; | |
| | Tinosorb S | |
| 46 | 1H-Benzimidazole-4,6-disulfonic acid, 2,2′-(1,4- | 180898-37-7 |
| | phenylene)bis-, disodium salt | |
| 47 | Benzoic acid, 4,4′-[[6-[[4-[[(1,1-dimethylethyl) | 154702-15-5 |
| | amino]carbonyl]phenyl]amino]1,3,5-triazine-2,4- | |
| | diyl]diimino]bis-, bis(2-ethylhexyl)ester; | |
| | diethylhexyl butamido triazone; Uvasorb HEB | |
| 48 | Phenol, 2-(2H-benzotriazol-2-yl)-4-methyl-6-[2- | 155633-54-8 |
| | methyl-3-[1,3,3,3-tetramethyl-1- | |
| | [(trimethylsilyl)oxy]disiloxanyl]propyl]-; | |
| | drometrizole trisiloxane; Mexoryl XL | |
| 49 | Dimethicodiethylbenzalmalonate; Polysilicone 15; | 207574-74-1 |
| | Parsol SLX | |
| 50 | Benzenesulfonic acid, 3-(2H-benzotriazol-2-yl)-4- | 92484-48-5 |
| | hydroxy-5-(1-methylpropyl)-, monosodium salt; | |
| | Tinogard HS | |
| 51 | Benzoic acid, 2-[4-(diethylamino)-2- | 302776-68-7 |
| | hydroxybenzoyl]-, hexyl ester; Uvinul a plus | |
| 52 | 1-Dodecanaminium, N-[3-[[4- | 156679-41-3 |
| | (dimethylamino)benzoyl]amino]propyl]-N,N- | |
| | dimethyl-, salt with 4-methylbenzenesulfonic | |
| | acid (1:1); Escalol HP610 | |
| 53 | 1-Propanaminium, N,N,N-trimethyl-3-[(1- | 177190-98-6 |
| | oxo-3-phenyl-2-propenyl)-amino]-, chloride | |
| 54 | 1H-Benzimidazole-4,6-disulfonic acid, 2,2′- | 170864-82-1 |
| | (1,4-phenylene)bis- | |
| 55 | 1,3,5-Triazine, 2,4,6-tris(4-methoxyphenyl)- | 7753-12-0 |
| 56 | 1,3,5-Triazine, 2,4,6-tris[4-[(2- | 208114-14-1 |
| | ethylhexyl)oxy]phenyl]- | |
| 57 | 1-Propanaminium, 3-[[3-[3-(2H-benzotriazol-2- | 340964-15-0 |
| | yl)-5-(1,1-dimethylethyl)-4-hydroxyphenyl]- | |
| | 1-oxopropyl]amino]-N,N- | |
| | diethyl-N-methyl-, methyl sulfate (salt) | |
| 58 | 2-Propenoic acid, 3-(1H-imidazol-4-yl)- | 104-98-3 |
| 59 | Benzoic acid, 2-hydroxy-, [4-(1- | 94134-93-7 |
| | methylethyl)phenyl]methyl ester | |
| 60 | 1,2,3-Propanetriol, 1-(4-aminobenzoate); | 136-44-7 |
| | glyceryl PABA | |
| 61 | Benzeneacetic acid, 3,4-dimethoxy-a-oxo- | 4732-70-1 |
| 62 | 2-Propenoic acid, 2-cyano-3,3-diphenyl-, ethyl ester | 5232-99-5 |
| 63 | Anthralinic acid, p-menth-3-yl | 134-09-8 |
| | ester | |
| 64 | 2,2′-bis(1,4-phenylene)-1H-benzimidazole- | 349580-12-7 |
| | 4,6-disulphonic acid mo- | |
| | no sodium salt or Disodium phenyl dibenzimidazole | |
| | tetrasulfonate or Neoheliopan AP | |
| 65 | 1,3,5-Triazine-2,4,6-triamine, N,N′-bis[4-[5-(1,1- | 288254-16-0 |
| | dimethylpropyl)-2-benzoxazolyl]phenyl]-N″- | |
| | (2-ethylhexyl)- or Uvasorb K2A | |
| 66 | Merocyanine derivatives as described in | |
| | WO 2004006878 and in IPCOM000022279D | |
| 67 | 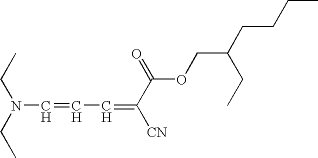 | |
| 68 | sterols (cholesterol, lanosterol, phytosterols), as | |
| | described in WO0341675 | |
| 69 | mycosporines and/or mycosporine-like amino acids | |
| | as described in WO2002039974, e.g. Helioguard | |
| | 365 from Milbelle AG, isolated mycosporine like | |
| | amino acids from the red alga porphyra umbilicalis | |
| | (INCI: Porphyra Umbilicalis) that are encapsulated | |
| | into liposomes,) | |
| 70 | alpha-lipoic-acid as described in DE 10229995 | |
| 71 | synthetic organic polymers as described in | |
| | EP 1371358, [0033]-[0041] | |
| 72 | phyllosilicates as described in EP 1371357 [0034]- | |
| | [0037] | |
| 73 | silica compounds as described in EP1371356, | |
| | [0033]-[0041] | |
| 74 | inorganic particles as described in DE10138496 | |
| | [0043]-[0055] | |
| 75 | latex particles as described in DE10138496 [0027]- | |
| | [0040] | |
| 76 | 1H-Benzimidazole-4,6-disulfonic acid, 2,2′- | 180898-37-7 |
| | (1,4-phenylene)bis-, disodium salt; Bisimidazylate; | |
| | Neo Heliopan APC | |

The topically applicable formulations of the present invention comprise at least a water phase and an oil-phase.

They are preferably formulated as water-in-oil (w/o) or water-in-silicone (w/Si) emulsions or microemulsions.

They may contain low molecular weight emulsifiers selected from non-ionic, cationic, amphoteric and anionic emulsifiers from 0.1 to 20% bw.

The UV-photoprotecting composition (c) is prepared by incorporating the primary C₆-C₂₈ fatty alcohol (componente (c₂)) into the UV absorber dispersion which contains the micronized UV absorber by simply mixing the 2 components.

The so obtained UV-photoprotecting composition (c) is preferably incorporated into the oil phase of the W/O emulsion.

The simple adding of such UV-photoprotecting composition helps to stabilize or even eliminate the crystal growth of the selected micronized organic UV-screening agent within the oil phase of the W/O or W/Si emulsion, or int the Si-phase of the w/Si emulsion respectively.

The final W/O or W/Si formulations according to the present invention may exist in a wide variety of presentation forms, for example:
in the form of liquid preparations as a W/O, O/W, O/W/O, W/O/W or PIT emulsion and all kinds of microemulsions,
in the form of a gel,
in the form of an oil, a cream, milk or lotion,
in the form of a stick,
in the form of a spray (spray with propellent gas or pump-action spray) or an aerosol,
in the form of a foam, or
in the form of a paste.

Of special importance as cosmetic preparations for the skin are light-protective preparations, such as sun milks, lotions, creams, oils, sunblocks or tropicals, pretanning preparations or after-sun preparations, also skin-tanning preparations, for example self-tanning creams. Of particular interest are sun protection creams, sun protection lotions, sun protection milk and sun protection preparations in the form of a spray.

Of special importance as cosmetic preparations for the hair are the above-mentioned preparations for hair treatment, especially hair-washing preparations in the form of shampoos, hair conditioners, hair-care preparations, e.g. pretreatment preparations, hair tonics, styling creams, styling gels, pomades, hair rinses, treatment packs, intensive hair treatments, hair-straightening preparations, liquid hair-setting preparations, hair foams and hairsprays. Of special interest are hair-washing preparations in the form of shampoos.

As water- and oil-containing emulsions or microemulsions the preparations contain, for example,
from 0.1 to 30% by weight, preferably from 0.1 to 15% by weight and especially from 0.5 to 10% by weight, based on the total weight of the composition, of an effective UV-photoprotecting composition (=component (c));
from 1 to 60% by weight, especially from 5 to 50% by weight and preferably from 10 to 35% by weight, based on the total weight of the composition, of at least one oil component,
from 0 to 30% by weight, especially from 1 to 30% by weight und preferably from 4 to 20% by weight, based on the total weight of the composition, of at least one emulsifier,
from 10 to 90% by weight, especially from 30 to 90% by weight, based on the total weight of the composition, of water, and
from 0 to 88.9% by weight, especially from 1 to 50% by weight, of further cosmetically acceptable adjuvants.

The topically applicable water-in-oil (w/o) or water-in-silicone (w/si) formulation may also contain one or one more additional compounds as esters of fatty acids,natural or synthetic triglycerides including glyceryl esters and derivatives, pearlescent waxes, hydrocarbon oils, silicones or siloxanes (organosubstituted polysiloxanes), fluorinated or perfluorinated oils, super-fatting agents, surfactants, consistency regulators/thickeners and rheology modifiers, polymers, biogenic active ingredients, deodorising active ingredients, anti-dandruff agents, antioxidants, hydrotropic agents, preservatives and bacteria-inhibiting agents, perfume oils, colourants, insect repellents or polymeric beads or hollow spheres as spf enhancers.

Cosmetic or pharmaceutical formulations are contained in a wide variety of cosmetic preparations like skin-care preparations, bath preparations, cosmetic personal care preparations, foot-care preparations, light-protective preparations, skin-tanning preparations, depigmenting preparations, insect-repellents, deodorants preparations for cleansing and caring for blemished skin, e.g. synthetic detergents (solid or liquid), peeling or scrub preparations or peeling masks; hair-removal preparations in chemical form (depilation),—shaving preparations, fragrance preparations, cosmetic hair-treatment preparations,

The cosmetic preparation according to the invention is distinguished by excellent protection of human skin against the damaging effect of sunlight.

### EXAMPLES

### Example 1

### W/O SunscreenContaining TINOSORB®M & Tinosorb®OMC

| | | Composition | |
|---|---|---|---|
| | | Ex. 1a | Ex. 1b |
| | | % w/w | % w/w |
| | INCI-Name | (as supplied) | (as supplied) |
| Part A | Glyceryl Oleate | 4.50 | 4.50 |
| | PEG-7 Hydrogenated Castor Oil | 1.50 | 1.50 |
| | Hydrogenated Castor Oil | 0.50 | 0.50 |
| | Microcrystalline Wax | 2.00 | 2.00 |
| | Ethylhexyl Methoxycinnamate | 5.00 | 5.00 |
| | Beeswax | 1.50 | 1.50 |
| | C12-15 Alkyl Benzoate | 8.00 | 8.00 |
| | Isopropyl Isostearate | 5.00 | 5.00 |
| | Mineral Oil | 5.00 | 5.00 |
| Part B | Aqua | qs to 100 | qs to 100 |
| | Magnesium Sulfate | 0.50 | 0.50 |
| | Citric Acid | 0.05 | 0.05 |
| Part C | Methylene Bis-Benzotriazolyl | 3.75 a.i. | 3.75 a.i. |
| | Tetramethylbutylphenol (and) | | |
| | Aqua (and) Propylene Glycol | | |
| | (and) Decyl Glucoside (and) | | |
| | Xanthan Gum | | |
| | Butyloctanol | 1.40 | — |
| | Blend of Octanol and Decanol | — | 0.90 |

Manufacturing instruction: Heat separately part A to 80° C. and B to 80° C. Slowly add part B to part A under high stirring. Decrease the speed till moderate stirring 500 rpm. Let cool down to 35° C. and add part C.

### Example 2

### W/O-Sunscreen

| | | Composition | |
|---|---|---|---|
| | | Ex. 2a | Ex. 2b |
| | | % w/w | % w/w |
| | INCI-Name | (as supplied) | (as supplied) |
| Part A | PEG-30 Dipolyhydroxystearate | 2.00 | 2.00 |
| | Cetearyl Isononanoate | 5.00 | 5.00 |
| | Hydrogenated Polydecene | 3.50 | 3.50 |
| | Ethylhexyl Methoxycinnamate | 5.00 | 5.00 |
| | C12-15 Alkyl Benzoate | 4.00 | 4.00 |
| | Cyclomethicone | 3.00 | 3.00 |
| Part B | Aqua | qs to 100 | qs to 100 |
| | Sorbeth-30 | 3.00 | 3.00 |
| | Magnesium Sulfate | 0.70 | 0.70 |
| Part C | Propylene Glycol (and) | 0.30 | 0.30 |
| | Diazolidinyl Urea (and) | | |
| | Methylparaben (and) | | |
| | Propylparaben | | |
| | Methylene Bis-Benzotriazolyl | 3.75 a.i. | 3.75 a.i. |
| | Tetramethylbutylphenol (and) | | |
| | Aqua (and) Propylene Glycol | | |
| | (and) Decyl Glucoside (and) | | |
| | Xanthan Gum | | |
| | Butyloctanol | 2.80 | — |
| | Blend of Octanol and Decanol | — | 1.40 |

Manufacturing instruction: Heat part A and part B separately to 80 C under stirring. Add part B to part A with continuous stirring. Homogenize for 30 sec with Ultra Turrax at 11000 rpm. Let cool down to 40° C. and add part C. Cool down to room temperature under continuous stirring.

### Example 3

### W/O Sunscreen

| | | Composition | |
|---|---|---|---|
| | | Ex. 3a | Ex. 3b |
| | | % w/w | % w/w |
| | INCI-Name | (as supplied) | (as supplied) |
| Part A | PEG-30 Dipolyhydroxystearate | | 5.00 |
| | Cetearyl Isononanoate | | 2.50 |
| | Hydrogenated Polydecene | | 6.50 |
| | Ethylhexyl Methoxycinnamate | | 4.00 |
| | C12-15 Alkyl Benzoate | | 0.50 |
| Part B | Aqua | | Qs to 100 |
| | Sorbeth-30 | | 1.00 |
| Part C | Propylene Glycol (and) | | 3.75 a.i. |
| | Diazolidinyl Urea (and) | | |
| | Methylparaben (and) | | |
| | Propylparaben | | |
| | Methylene Bis-Benzotriazolyl | | 0.30 |
| | Tetramethylbutylphenol (and) | | |
| | Aqua (and) Propylene Glycol | | |
| | (and) Decyl Glucoside (and) | | |
| | Xanthan Gum | | |
| | Butyloctanol | 1.15 | — |
| | Blend of Octanol and Decanol | — | 1.15 |

Appearance: white viscous emulsion
Viscosity (Brookfield DVIII+LV/T-C/50 rpm): 80000-100000 mPas
   Manufacturing instruction: Heat part A to 70° C. under continuous stirring. Add part B to part A and homogenize for a minute by 11000 rpm. Let cool down to room temperature and add part C under gentle stirring

### Example 4

### W/O Sun Emulsion

| | | Composition | |
|---|---|---|---|
| | | Ex. 4a | Ex. 4b |
| | | % w/w | % w/w |
| | INCI-Name | (as supplied) | (as supplied) |
| Part A | Sorbitan Isostearate | 5.00 | 5.00 |
| | Lanolin Alcohol | 1.00 | 1.00 |
| | PEG-7 Hydrogenated Castor Oil | 1.50 | 1.50 |
| | Microcrystalline Wax | 1.50 | 1.50 |
| | Vaseline | 3.00 | 3.00 |
| | Caprylic/Capric Triglyceride | 1.50 | 1.50 |
| | Ethylhexyl Methoxycinnamate | 5.00 | 5.00 |
| | Paraffinium Subliquidum | 5.00 | 5.00 |
| Part B | Aqua | qs to 100 | qs to 100 |
| | Magnesium Sulfate | 0.70 | 0.70 |
| Part C | Methylene Bis-Benzotriazolyl | 3.75 a.i. | 3.75 a.i. |
| | Tetramethylbutylphenol (and) | | |
| | Aqua (and) Propylene Glycol | | |
| | (and) Decyl Glucoside (and) | | |
| | Xanthan Gum | | |
| | Propylene Glycol (and) | 0.30 | 0.30 |
| | Diazolidinyl Urea (and) | | |
| | Methylparaben (and) | | |
| | Propylparaben | | |
| | Butyloctanol | 0.80 | |
| | Blend of Octanol and Decanol | | 0.80 |

* measurement made on Labsphere UV Transmittance Analyzer 1.2 μl/cm² on PMMA

Manufacturing instruction: Heat phase A to 75° C. Add part B to part A and homogenize for 30 sec. at 11000 rpm. Let cool down to 50° C. and add part C. Continuous stirring until room temperature.

1. A topically applicable water-in-oil or water-in-silicone formulation comprising:
(a) at least one aqueous phase;
(b) at least one fatty phase;
(c) an effective UV-photoprotecting composition comprising
(c₁) 20% to 99% of at least one insoluble micronized organic UV-screening active with a mean particle size ranging from 0.01 micrometers to 2 micrometers; and
(c₂) 0.5% to 80% of at least a primary C₆-C₂₈ fatty alcohol.

2. A formulation according to claim 1, wherein
(c₁) is selected from a triazine, a benzotriazole, a benzophenone, a vinyl group-containing amide, a cinnamic acid amide and a sulfonated benzimidazole UV absorber.

3. A formulation according to claim 2, wherein the triazine UV absorber is a compound of the formula 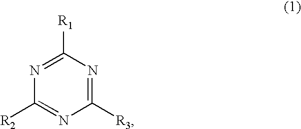
wherein
R₁, R₂ and R₃, independently from each other, are hydrogen; hydroxy; C₁-C₃alkoxy; NH₂; NHR₄; N(R₄)₂; OR₄; C₆-C₁₂aryl; phenoxy; anilino; pyrrolo; in which the respective phenyl, phenoxy, anilino or pyrrolo moieties are not substituted or substituted by one, two or three substitutents selected from OH, carboxy, CO—NH₂, C₁-C₁₂alkyl, C₁-C₁₂alkoxy, a methylidenecamphor group, a group—(CH═CH)ₘC(═O)—OR₄, a group 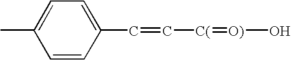
or the corresponding alkali metal, ammonium, mono-, di- or tri-C₁-C₄alkylammonium, mono-, di- or tri-C₂-C₄alkanolammonium salts, or the C₁-C₃alkyl esters thereof or by a radical of formula 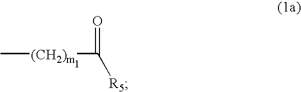
R₄ is C₁-C₅alkyl;
R₅ is hydroxy; C₁-C₅alkyl that is unsubstituted or substituted by one or more OH groups; C₁-C₅alkoxy; amino; mono- or di-C₁-C₅alkylamino; M; a radical of formula 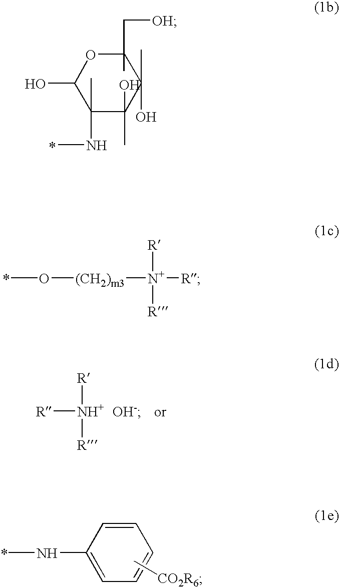
R′, R″ and R′″ independently of the other are C₁-C₁₄alkyl that is unsubstituted or substituted by one or more OH groups;
R₆ is hydrogen; M; C₁-C₅alkyl; or a radical of the formula 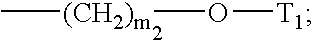
M is a metal cation;
T₁ is hydrogen; or C₁-C₈alkyl;
m is 0 or 1;
m₁ is from 1 to 5;
m₂ is from 1 to 4; and
m₃ is from 2 to 14.

4. A formulation according to claim 2, wherein
R₁, R₂ and R₃ independently from each other are a radical of formula 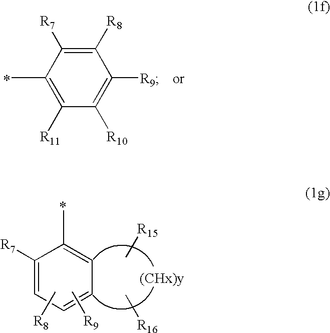
R₇ and R₁₁ independently from each other are hydrogen; C₁-C₁₈alkyl; or C₆-C₁₂aryl;
R₈, R₉ and R₁₀, independently from each other, are hydrogen; or a radical of formula 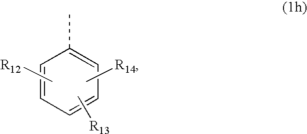
wherein, in formula (1f), at least one of the radicals R₈, R₉ and R₁₀ are a radical of formula (1h);
R₁₂, R₁₃, R₁₄, R₁₅ and R₁₆ independently from each other are hydrogen; hydroxy; halogen; C₁-C₁₈alkyl; C₁-C₁₈alkoxy; C₆-C₁₂aryl; biphenylyl; C₆-C₁₂aryloxy; C₁-C₁₈alkylthio; carboxy; —COOM; C₁-C₁₈-alkylcarboxyl; aminocarbonyl; mono- or di-C₁-C₁₈alkylamino; C₁-C₁₀acylamino; or —COOH;
M is an alkali metal ion;
x is 1 or 2; and
y is a number from 2 to 10.

5. A formulation according to claim 4, wherein the triazine compound is of formula 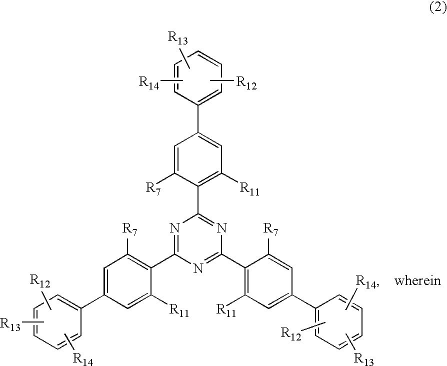
R₇, R₁₁, R₁₂, R₁₃ and R₁₄ are defined as in claim 4.

6. A formulation according to claim 5, wherein
R₇ and R₁₁ are hydrogen.

7. A formulation according to claim 4, wherein component (c₁) is a compound of formula 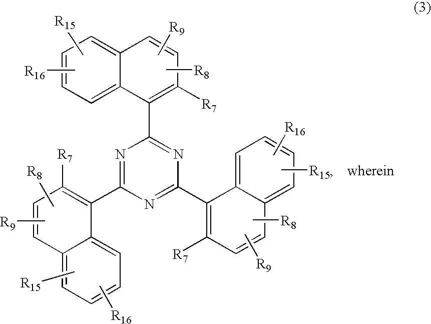
R₇, R₈, R₉, R₁₅ and R₁₆ are defined as in claim 4.

8. A formulation according to claim 7, wherein
R₇, R₈, R₉, R₁₅ and R₁₆ are hydrogen; or, independently from each other, C₁-C₁₈alkyl.

9. A formulation according to claim 5, wherein component (c₁) is a compound of formula

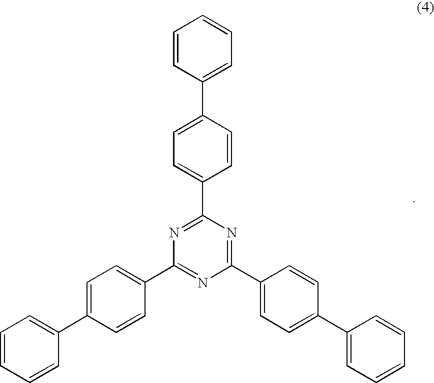

10. A formulation according to claim 2, wherein the triazine UV absorber is a compound of formula 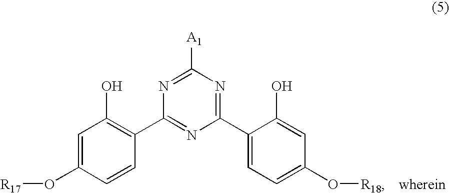
R₁₇ and R₁₈, independently of one another, are C₁-C₁₈alkyl; C₂-C₁₈alkenyl; a radical of the formula —CH₂—CH(—OH)—CH₂—O-T₁; or a radical of the formula —(CH₂)ₘ_{₁}—O—(CH₂)ₘ_{₂}-T₂; a radical of the formula 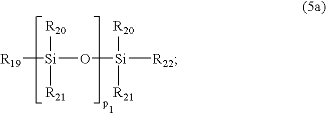
R₁₉is a direct bond; a straight-chain or branched C₁-C₄alkylene radical or a radical of the formula —Cₘ_{₁}H₂ₘ_{₁}— or —Cₘ_{₁}H₂ₘ_{₁}—O—;
R₂₀, R₂₁ and R₂₂, independently of one another, are C₁-C₁₈alkyl; C₁-C₁₈alkoxy or a radical of the formula 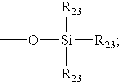
R₂₃ is C₁-C₅alkyl;
T₁ and T₂, independently from each other, are hydrogen; or C₁-C₈alkyl;
m₁, m₂ and m₃, independently of one another, are 1 to 4;
p₁ is 0; or a number from 1 to 5;
A₁ is a radical of the formula 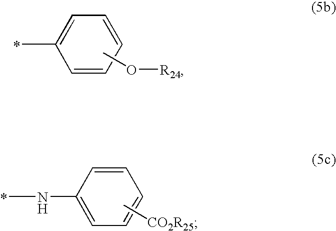
or of the formula 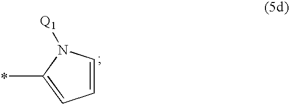
R₂₄ is hydrogen; C₁-C₁₀alkyl, —(CH₂CHR₂₆—O)ₙ_{₁}—R₂₅; a —CH₂—CH(—OH)—CH₂—O-T₁; or radical of the formula —(CH₂)ₘ_{₁}—O—(CH₂)ₘ_{₂}T₂;
R₂₅ is hydrogen; M; C₁-C₅alkyl; or a radical of the formula —(CH₂)ₘ_{₂}—O-T₁;
R₂₆ is hydrogen; or methyl;
Q₁ C₁-C₁₈alkyl;
M is a metal cation; and
n₁ is 1-16.

11. A sun screen formulation according to claim 2 wherein the triazine compound according to component (c₁) is a compound of formula 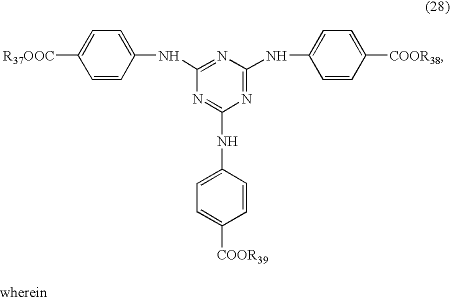
R₃₇, R₃₈ and R₃₉, independently from each other are hydrogen; an alkali metal; an ammonium group N⁺(R₄₀)₄;
R₄₀ is hydrogen; or an organic radical; C₁-C₃alkyl; or a polyoxyethylene radical which contains from 1 to 10 ethylene oxide units and the terminal OH group of which may be etherified by a C₁-C₃alcohol.

12. A formulation according to claim 2 wherein the benzotriazole organic UV absorber is of the formula 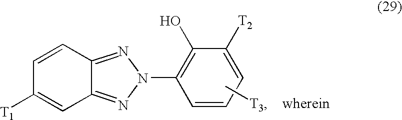
T₁ is C₁-C₃alkyl or, preferably, hydrogen; or a radical of formula 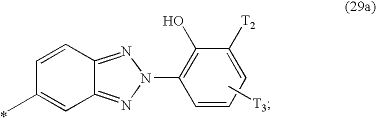
and
T₂ and T₃, independently from each other are C₁-C₁₂alkyl, preferably i-octyl; or C₁-C₄alkyl substituted by phenyl, preferably α,α-dimethylbenzyl.

13. A formulation according to claim 2 wherein the benzotriazole organic UV absorber is a compound of formula 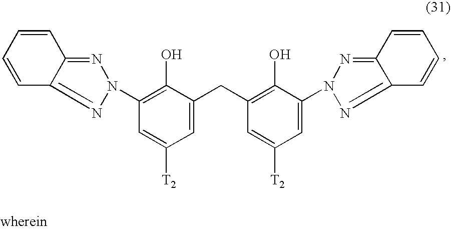
T₂ is hydrogen; C₁-C₁₂alkyl; or C₁-C₄alkyl substituted by phenyl.

14. A formulation according to claim 13 wherein
T₂ is iso-octyl.

15. A formulation according to claim 1, wherein
(c₂) is a linear or linear branched C₈-C₂₀alcohol.

16. A formulation according to claim 15, wherein
(c₂) is a compound of the formula (35) 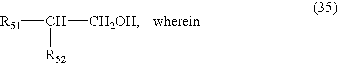
R₅₁ and R₅₂, independently from each other, are a linear C₁-C₁₈alkyl, wherein the sum of carbon atoms in R₅₁ and R₅₂ is less than or equal to 18.

17. A formulation according to claim 16, wherein the fatty alcohol is selected from the group consisting of compounds of formulae

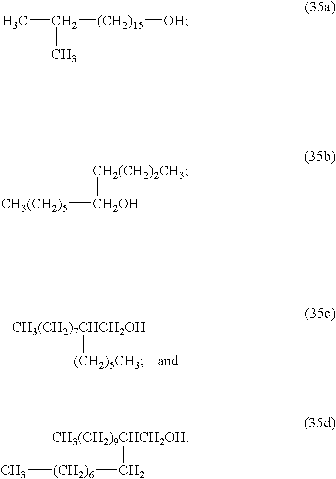

18. A formulation according to claim 15 wherein
(c₂) is a mixture of linear octanol and linear decanol.

19. A formulation according to claim 1 wherein the sun screen formulation additionally comprises an oil-soluble organic UV absorber selected from the group consisting of a p-aminobenzoic acid derivative; a salicylic acid derivative; a benzophenone derivative; a dibenzoylmethane derivative; a diphenylacrylate derivative; a benzofuran derivative; a polymeric UV absorber containing one or more silico-organic residues; a cinnamate ester; a camphor derivative; a trianilino-s-triazine derivative; phenylbenzimidazole sulfonic acid or one of its salts; urocanic acid (3-imidazol-4-yl-acrylic acid) or its ethyl ester; menthyl anthranilate; a benzotriazole; a hydroxyphenyltriazine derivative; and a bis-resorcinol-dialkylaminotriazine.
